(19) United States
(12) Patent Application Publication
(10) Pub. No.: US 2008/0167541 A1
(43) Pub. Date: Jul. 10, 2008

(54) Interference Suppression in Spectral Plethysmography

(76) Inventors: Panu Takala, Helsinki (FI); Borje Rantala, Helsinki (FI)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/621,203

(22) Filed: Jan. 9, 2007

(51) Int. Cl. A61B 5/1455

(52) U.S. Cl. 600/323

(57) ABSTRACT

The invention relates to a patient monitoring device, especially to a pulse oximeter. For suppressing the effects of an interference source causing adverse modulation in one or more blood related signals input to the device, a power spectrum is derived for each blood related signal and the frequency of an interference source causing unwanted modulation in the blood related signal(s) is identified. A weight function is determined, in which the relative weight of at least one frequency is dampened, the at least one frequency depending on the identified frequency. The weight function is applied to the power spectrum(s), thereby to obtain at least one weighted power spectrum. A blood related parameter of the subject is defined based on the at least one weighted power spectrum.

### FIELD OF THE INVENTION

The present invention relates generally to assessment of a blood related physiological parameter in a subject. More particularly, the present invention relates to a mechanism for suppressing the effects of an interference source causing adverse modulation in one or more blood related signals input to a monitoring device in which spectral representation(s) of the blood related signal(s) is/are determined. In a typical application, the device is a pulse oximeter, while the interference source is the respiration of the subject. The blood related signal may be a plethysmographic or a blood pressure signal.

### BACKGROUND OF THE INVENTION

Plethysmography refers to measurement of changes in the sizes and volumes of organs and extremities by measuring changes in blood volume. Photoplethysmography relates to the use of optical signals transmitted through or reflected by blood for monitoring a physiological parameter of a subject. Pulse oximeters use red and infrared photoplethysmographic (PPG) waveforms to determine oxygen saturation of pulsatile arterial blood and cardiac pulse rate of a subject. Pulse oximetry is at present the standard of care for continuous monitoring of arterial oxygen saturation (SpO₂). Pulse oximeters provide instantaneous in-vivo measurements of arterial oxygenation, and thereby an early warning of arterial hypoxemia, for example. Pulse oximeters also display a photoplethysmographic pulse waveform, which can be related to tissue blood volume and blood flow, i.e. the blood circulation, at the site of the measurement, typically in finger or ear.

A pulse oximeter comprises a computerized measuring unit and a sensor attached to the patient, typically to a finger or ear lobe. The sensor includes a light source for sending an optical signal through the tissue and a photo detector for receiving the signal after transmission through the tissue. On the basis of the transmitted and received signals, light absorption by the tissue can be determined. During each cardiac cycle, light absorption by the tissue varies cyclically. During the diastolic phase, absorption is caused by arterial and venous blood, tissue, bone, and pigments, whereas during the systolic phase there is an increase in absorption, which is caused by the influx of arterial blood into the tissue. Pulse oximeters focus the measurement on this arterial blood portion by determining the difference between the peak absorption during the systolic phase and the constant absorption during the diastolic phase. Pulse oximetry is thus based on the assumption that the pulsatile component of the absorption is due to arterial blood. Light transmission through an ideal absorbing sample is determined by the known Lambert-Beer equation as follows:

Iₒᵤₜ=Iᵢₙe^{-εDC}, (1)

where Iᵢₙ is the light intensity entering the sample, Iₒᵤₜ is the light intensity received from the sample, D is the path length through the sample, ε is the extinction coefficient of the analyte in the sample at a specific wavelength, and C is the concentration of the analyte. When Iᵢₙ, D, and ε are known, and Iₒᵤₜ is measured, the concentration C can be calculated.

In pulse oximetry, in order to distinguish between two species of hemoglobin, oxyhemoglobin (HbO₂), and deoxyhemoglobin (RHb), absorption must be measured at two different wavelengths, i.e. the sensor normally includes two different light emitting diodes (LEDs). The wavelength values widely used are 660 nm (red) and 940 nm (infrared), since the said two species of hemoglobin have substantially different absorption values at these wavelengths. Each LED is illuminated in turn at a frequency which is typically several hundred Hz.

Based on their physiological origin, both red and infrared plethysmographic waveforms can be divided into two components, AC and DC. The DC component is a low frequency portion of the signal, which generally corresponds to the light absorption in the non-pulsatile volume of the perfused tissue. Ideally, the AC component represents a varying waveform originating from the pulsatile arterial blood, and it's frequency corresponds to that of the heartbeat. The ratio of the AC component to the DC component (AC/DC) defines the modulation percentage a plethysmographic signal.

In addition to arterial pulsation, the plethysmographic waveform may be affected by other sources, such the respiration of the subject. In terms of determining the SpO₂ of the subject, the time-varying components that are not caused by the arterial pulsation are regarded as interference.

The oxygen saturation value is usually calculated by defining the ratio of the red and infrared AC/DC ratios and obtaining the saturation value from an empirically determined calibration curve. The numeric value of the ratio R of red to infrared normalized AC signals is related to arterial oxygen saturation and can be obtained as follows:

R=(AC_{red}/DC_{red})/(AC_{ired}/DC_{ired}).

Pulse oximeters use an empirically determined calibration curve f to transform R values into SpO₂ percentage:

SpO₂=f(R).

Another frequently used method for calculating the SpO₂ value is to transform the plethysmographic signals into frequency domain and use the power spectral density (PSD) of the red and infrared signals to calculate a numeric value for R. In this method, the AC components of both red and infrared plethysmographic waveforms are first divided by the corresponding DC component in time-domain. The DC normalized AC signals obtained are then transferred into frequency domain by applying a Fast Fourier Transform (FFT), for example, to the time-domain signals to obtain the power spectrum corresponding to each signal. The power spectrum shows the power distribution of the corresponding normalized waveform as a function of frequency. Ideally, when no noise is present in the plethysmographic signals, both the red and infrared power spectra have a peak value at the frequency of the heart rate. These peaks are then used to calculate R as follows:

R=sqrt(PSD_{—}i(AC_{red}/DC_{red})/PSD_{—}i(AC_{ired}/DC_{ired})) (2),

where sqrt refers to square root and PSD_i refers to signal power values at the frequency of heart rate pulsation. The SpO₂ value is then obtained by using the same calibration curve f as in the time-domain analysis.

If the arterial pulsation were purely sinusoidal, the power spectra would have a single peak at the heart rate. In practice, however, the plethysmographic waveform differs in morphology from that of a sine wave. Therefore, the arterial pulsation gives rise to not only one, but several power spectrum peaks located at harmonic frequencies of the pulse rate PR, i.e. at frequencies N*PR, where N=1, 2, 3 . . . .

The determination of SpO₂ values in frequency domain is disclosed in U.S. Pat. No. 4,934,372, for example.

As mentioned above, the plethysmographic waveform obtained in a pulse oximeter usually also comprises components caused by other sources than the heart. One such component may originate from the respiratory function of the patient. The intrathoracic pressure of a spontaneously breathing subject decreases during inspiration and increases during expiration. The pressure variations are transferred to the central venous pressure (CVP). This, in turn, leads to elevated venous return (VR) to the right atrium of the heart during inspiration and to a minor decrease in VR during expiration. In patients ventilated with positive pressure ventilation, pressure variations are reversed. Consequently, inflation of the lungs causes elevation of intrathoracic pressure and a decrease in VR.

Respiratory variation of VR makes the peripheral venous pressure (PVP) fluctuate with respiration. During spontaneous inspiration, blood flow towards thorax is facilitated and the veins are drained. In a photoplethysmogram, the drainage can be seen as an increase in the signal amplitude, due to a decrease in the absorption of the transmitted light. In expiration, VR is hindered and thus the increased amount of blood in the veins increases light absorption, which translates to a decrease in signal amplitude.

In time-domain, the respiratory variation described above can be seen as fluctuation of the baseline of the plethysmographic signal at the frequency of respiration. In the power spectrum of the plethysmographic signal, the respiratory-induced fluctuation can be seen as peaks at the respiration rate (RR) and the harmonic frequencies N*RR (N=2, 3 . . . ) thereof.

Consequently, a drawback related to the determination of SpO₂ values based on spectral representations of PPG signals is that pulsation induced by an interference source, such as respiration, may lead to a situation in which the frequency of the interference source, or a harmonic frequency thereof, is erroneously regarded as the pulse rate of the subject. This in turn leads to false SpO₂ readings. The risk of an erroneous pulse rate selection is higher if the amplitude of the interference-induced pulsation is high relative to that of arterial pulsation. In other words, the risk of false SpO₂ readings is generally higher in patients having low amplitude arterial pulsation.

The present invention seeks to eliminate or alleviate this drawback related to the determination of SpO₂ values in frequency domain.

### SUMMARY OF THE INVENTION

The present invention seeks to improve the reliability of the evaluation of a blood related parameter in devices utilizing the spectral representation(s) of one or more blood related signals, i.e. plethysmographic or blood pressure signals. The invention further seeks to reduce the probability of false SpO₂ calculations in presence of respiration related artefacts in the plethysmographic signals obtained from a subject.

In the present invention, adverse modulation produced by an interference source in a blood related signal is suppressed by identifying the frequency of the interference source and weighting the spectral representation of the blood related signal by a weighting function in which the relative weights of the frequencies are selected in dependence on the identified frequency. Adverse modulation here refers to modulation which is not caused by the arterial pulsation caused by the cardiac function of the subject. The interference source may thus be any intrinsic or extrinsic source that causes, in addition to the modulation caused by arterial pulsation, extra modulation in the blood related signal. The weighted power spectrum(s) is/are then used to evaluate the blood related parameter, such as arterial oxygen saturation, of the subject. Although the mechanism of the invention finds a typical application in pulse oximetry, the device of the invention may be used in any application in which the spectral representation of at least one blood related signal is determined. It is also to be noted here that the device is not necessarily a non-invasive measurement device; arterial blood pressure, for example, may be measured invasively using catheters inserted in the arteries or veins.

Thus one aspect of the invention is providing a method for measuring a blood related parameter of a subject. The method includes obtaining at least one blood related signal from a subject and deriving a power spectrum for at least one of the at least one blood related signal, thereby to obtain at least one power spectrum. The method also includes identifying at least one interference frequency of at least one interference source causing unwanted modulation in the at least one blood related signal and determining a weight function in which the relative weight of at least one frequency is dampened, the at least one frequency depending on the at least one interference frequency identified. The method further includes applying the weight function to at least one of the at least one power spectrum, thereby to obtain at least one weighted power spectrum and defining the blood related parameter based on the at least one weighted power spectrum.

The invention provides an uncomplicated mechanism for improving the reliability of spectral plethysmographs, since the weighting operation may easily be inserted into a standard patient monitoring device.

Another aspect of the invention is that of providing an apparatus for assessing a blood related parameter of a subject. The apparatus includes a measurement unit configured to measure at least one blood related signal from a subject, a transform unit configured to derive a power spectrum for at least one of the at least one blood related signal, thereby to obtain at least one power spectrum, and a frequency identification unit configured to identify at least one interference frequency of at least one interference source causing unwanted modulation in the at least one blood related signal. The apparatus further comprises a first calculation unit configured to determine a weight function in which the relative weight of at least one frequency is dampened, the at least one frequency depending on the at least one interference frequency, a weighting unit configured to apply the weight function to at least one of the at least one power spectrum, thereby to obtain at least one weighted power spectrum, and a second calculation unit configured to define the blood related parameter based on the at least one weighted power spectrum.

A still further aspect of the invention is that of providing a computer program product by means of which an existing monitoring device may be upgraded to carry out the suppression of adverse modulation in a plethysmographic signal. The computer product comprises a first program code portion configured to determine a weight function in which the relative weight of at least one frequency is dampened, the at least one frequency depending on at least one frequency of an interference source causing unwanted modulation in at least one blood related signal and a second program code portion configured to apply the weight function to at least one power spectrum of the at least one blood related signal, thereby to obtain at least one weighted power spectrum.

A further advantage of the invention is that it reduces the probability of a false pulse rate indication in the monitoring device, such as a pulse oximeter.

Other features and advantages of the invention will become apparent by reference to the following detailed description and accompanying drawings.

### BRIEF DESCRIPTION OF THE DRAWINGS

In the following, the invention and its preferred embodiments are described more closely with reference to the examples shown in FIGS. 1 to 9 in the appended drawings, wherein:

FIG. 1 is a block diagram illustrating one embodiment of a pulse oximeter according to the invention;

FIG. 2 illustrates the determination of SpO₂ values in frequency domain;

FIGS. 3a and 3b illustrate the determination of the ratio R in a pulse oximeter;

FIG. 4 illustrates the determination of the SpO₂ value based on the R value;

FIGS. 5a and 5b illustrate the effects of respiration in a plethysmographic signal;

FIG. 6 illustrates the selection of the pulse rate in one embodiment of the invention;

FIG. 7 illustrates one embodiment of the weight function of the invention;

FIG. 8 illustrates a further embodiment of the weight function of the invention; and

FIG. 9 illustrates the selection of the pulse rate in a further embodiment of the invention.

### DETAILED DESCRIPTION OF THE INVENTION

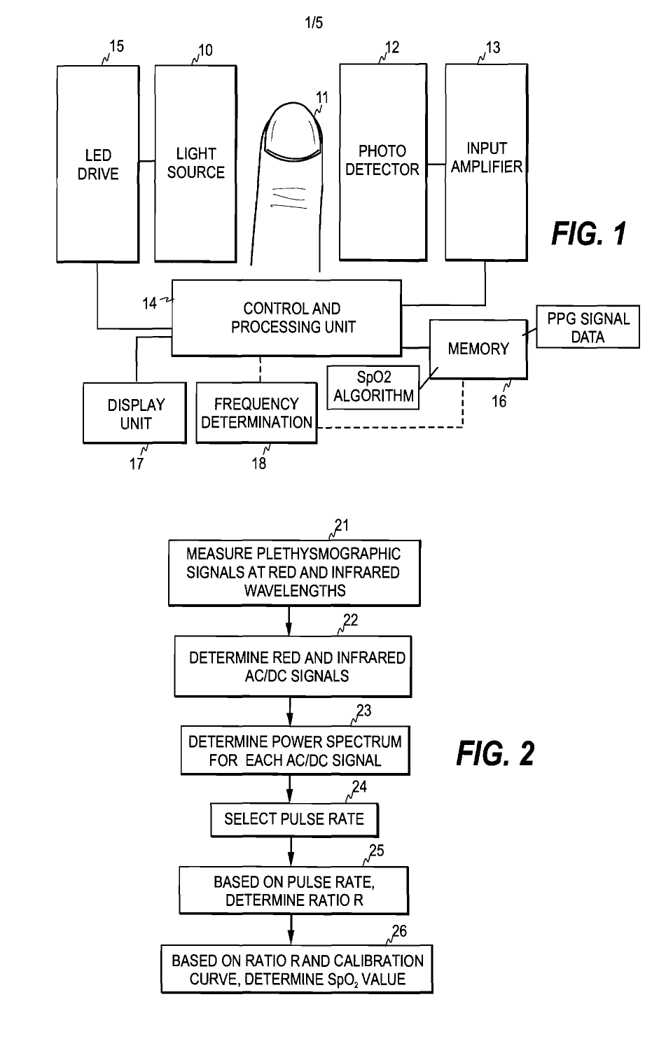

FIG. 1 is a block diagram of one embodiment of a pulse oximeter according to the invention. Light transmitted from a light source 10 including the LEDs passes into patient tissue, such as a finger 11. The light propagated through or reflected from the tissue is received by a photo detector 12, which converts the optical signal received into an electrical signal and feeds it to an input amplifier 13. The amplified signal is then supplied to a control and processing unit 14, which converts the signals into digitized format. The digitized signal data is then utilized by an SpO₂ algorithm for determining the blood oxygen saturation. The control and processing unit executes the algorithm and drives a display 17 to present the results on the screen of the display. The SpO₂ algorithm may be stored in a memory 16 of the control and processing unit. The digitized PPG signal data may also be stored in the said memory before being supplied to the SPO₂ algorithm.

The control and processing unit further controls a source drive 15 to alternately activate the LEDs. As mentioned above, a pulse oximeter typically includes one LED emitting at a red wavelength and one LED emitting at an infrared wavelength, and each LED is typically illuminated several hundred times per second. However, the number of emitter elements in a sensor depends on the application used. Plethysmographic data, for example, may be measured with one emitter element only (one wavelength), while a pulse oximeter may also include more than two emitter elements in each sensor.

With each LED being illuminated at such a high rate as compared to the pulse rate of the patient, the control and processing unit obtains a high number of samples at each wavelength for each cardiac cycle of the patient. The value of these samples (i.e. the amplitude of the received signal) varies according to the cardiac cycle of the patient, the variation being caused by the arterial blood, as mentioned above.

The pulse oximeter of the invention may further include a module 18 configured to determine the frequency of an interference source causing unwanted modulation in the plethysmographic signals. The module may be a software module residing in memory 16, or the pulse oximeter may be connected to an external measurement arrangement 18 supplying the frequency information to the pulse oximeter. The frequency of the interference source may be displayed on the screen of the display 17.

FIG. 2 is a flow diagram illustrating the operation of an SpO₂ algorithm in which the spectra of the plethysmographic signals are determined. It is thus assumed here that the device is a pulse oximeter comprising two emitter elements emitting respectively at red and infrared wavelengths. Upon obtaining the plethysmogrpahic signals from the subject (step 21), the control and processing unit determines the AC and DC components of light absorption at both wavelengths and divides each AC component by the respective DC component (step 22), thereby obtaining the AC/DC ratio for both PPG signals. The division of the plethysmographic signal data into the AC and DC components may be carried out by a filter. The normalization of the AC component makes the signal obtained widely independent of extrinsic factors, such as the brightness of the LEDs, the sensitivity of the detector, skin pigmentation, or the thickness of the finger or ear.

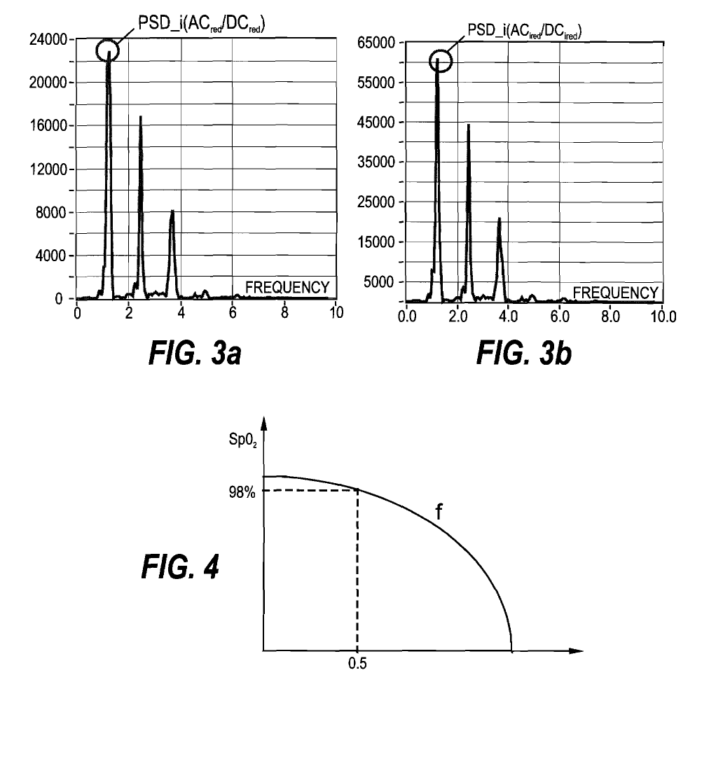

The red and infrared normalized AC signals (AC/DC) are then subjected to a Fourier transform at step 23, thereby to obtain the corresponding red and infrared power spectrums. FIGS. 3a and 3b show, respectively, examples of the red and infrared power spectrums of the normalized AC signals. In this example, the selection of the pulse rate is not impeded by an interference source. The peak corresponding to pulse rate can be seen at a frequency of 1.3 Hz, whereas other peaks occur at harmonic frequencies of the pulse rate.

Since the ratio R, which is uniquely related to arterial oxygen saturation, is calculated according to equation (2) above, the SpO₂ algorithm needs to find the spectrum peaks that correspond to the pulse rate in the red and infrared spectra in order to be able to determine the AC/DC values used in equation (2). The selection of the pulse rate is carried out in step 24 of FIG. 2. As the highest peak usually represents the pulse rate, conventional pulse oximeters normally select the highest peak as the peak representing the pulse rate.

However, as is discussed below, in a pulse oximeter according to the invention the selection of the pulse rate is not made directly based on the power spectrum values.

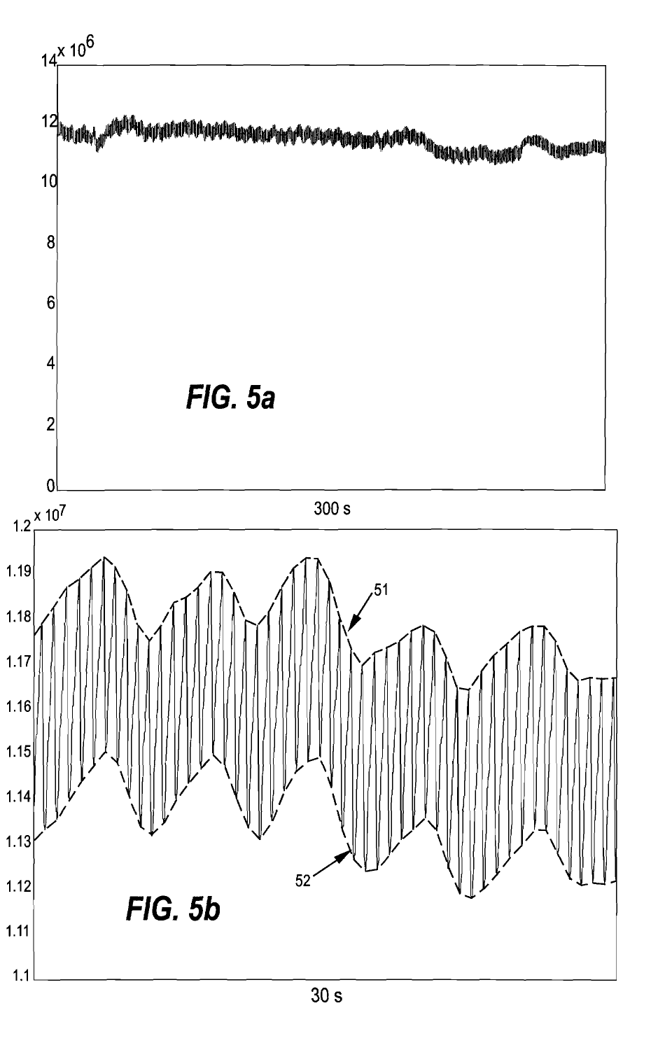

When the pulse rate has been selected, the numeric value of ratio R can be calculated according to equation (2) above (step 25), using the values of the selected peaks as the values of PSD_i(AC_{red}/DC_{red}) and PSD_i(AC_{ired}/DC_{ired}). An empirically determined calibration curve may then be used (step 26) to transform the obtained R value into an SPO₂ percentage. FIG. 4 shows an example in which the calibration curve f converts an R value of 0.5 to an SpO₂ value of 98%.

As discussed above, the respiration of the patient causes extra modulation in the plethysmographic signal and may thus disturb the selection of the pulse rate based on the power spectrum values. FIGS. 5a and 5b illustrate the effects of respiration in a plethysmographic signal. FIG. 5a illustrates a 5 minute interval of the signal, while FIG. 5b shows a period of 30 seconds within said 5 minute period. The modulation caused by the respiration of the patient may be seen in three different features of the plethysmographic signal. First, the amplitude of the component pulsating at the heart rate varies according to the respiration. This may be seen as a modulation in the difference of the top and bottom envelope curves, 51 respectively 52, of the signal in FIG. 5b. Second, the pulse-to-pulse interval, i.e. the interval between successive heart beats, varies according to respiration, the said interval being shorter during inspiration phases than during expiration phases. Third, the DC baseline signal, i.e. the top or bottom envelope of the plethysmographic signal, varies slowly according to the respiration.

To decrease the probability of the respiration frequency or the frequency of another interference source being erroneously selected as the pulse rate, the pulse rate of the subject is selected at step 24 of FIG. 2 according to weighted power spectrum values.

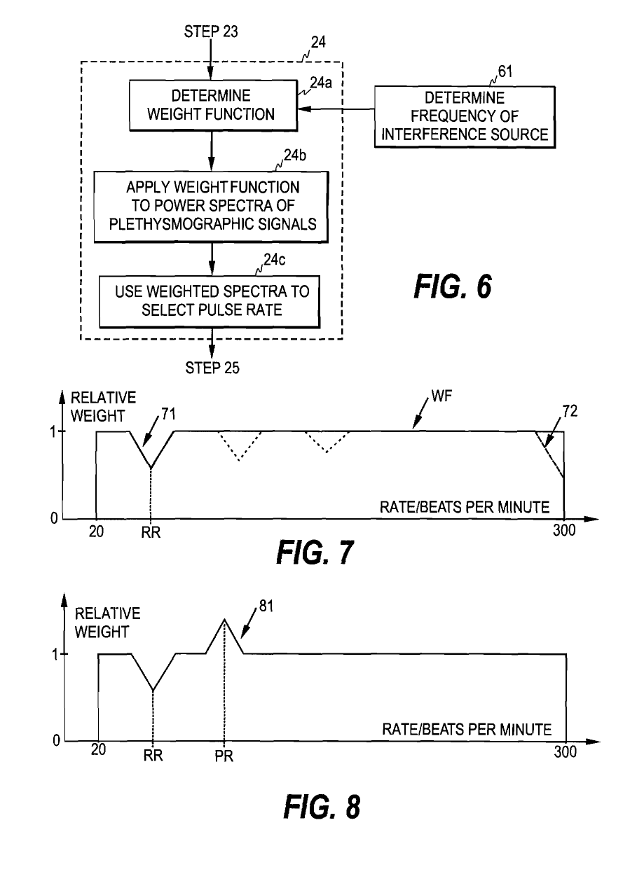

FIG. 6 illustrates one embodiment of the pulse rate selection of the invention. The selection rests on the identification of the frequency of an interference source concerned, which is carried out in step 61. As discussed above, the interference source is typically the respiration of the subject, although the mechanism of the invention may also be used for suppressing the unwanted modulation caused by sources other than respiration. The determination of the respiration rate of the subject may be performed by any appropriate method and various methods may be used depending on the equipment available. The parameters that may be used to determine the respiration rate include CO₂, airway pressure, or impedance respiration, for example. The respiration rate may also be extracted from PPG signal data in a known manner. U.S. Pat. Nos. 7,001,337 and 6,896,661 describe methods for extracting the respiration rate from a plethysmographic signal. If the respiration rate is lower than about 30 bpm and plethysmographic power spectrum peak at the respiration rate is high, the respiration rate may easily be determined from the spectrum if the heart rate is higher than 30 bpm. The plethysmographic respiratory peak is usually much higher than its harmonic peaks, which make the rejection of harmonics reliable. It is also possible that the nursing staff measures the respiration rate simply by counting the number of breaths in a time unit.

Based on the interference frequency, such as the respiration rate, the algorithm of the apparatus of the invention determines a weight function, in which the weight of the power spectrum values being close to the respiration rate and/or its harmonic components is diminished (step 24a), and the weight function is applied to the power spectrum values obtained from the Fourier transform (step 24b). The selection of the pulse rate may then be made based on the weighted power spectrum values (step 24c), i.e. the frequency corresponding to the highest weighted peak may be selected as the pulse rate.

FIG. 7 illustrates the weight function WF according to one embodiment of the invention. In this embodiment, the relative weight is zero below a lower limit of 20 bpm and beyond an upper limit of 300 bpm, i.e. all frequencies outside this passband are discarded. Furthermore, if the interference frequency is within the passband, the weight function further comprises a notch 71 around the interference frequency. Within the passband and outside the notch the relative weight is one, i.e. the said frequencies are neither dampened nor enhanced. The slopes of the passband edges may also vary.

In a further embodiment of the invention, the weight of those harmonic frequencies of the interference frequency, which locate between the lower limit and the upper limit, may also be dampened, as is illustrated by dashed notches in FIG. 7. If the respiration rate, for example, is below the lower limit, a notch may be made for one or more of its harmonic frequencies locating within the passband. The depth of the notches corresponding to the harmonics may be smaller than the depth of the notch corresponding to the respiration rate, as is illustrated in the figure.

Although its form, width, and depth may vary, the notch is preferably such that the weight function has a local minimum at the interference frequency. If respiration is the interference source, the width of the notch is typically approximately 10 bpm (beats per minute), in which case the lower and upper limits of the notch correspond respectively to frequencies (RR−5) bpm and (RR+5) bpm, where the respiration rate RR is measured in beats per minute. However, if the respiration rate or its harmonic frequency is near the lower or upper limit of the passband, the form of the weight function may change at the corresponding end of the passband according to the distance of the said frequency from the edge of the passband. For example, if the said distance is shorter than half of the width of the notch, the weight function may be sloped at the corresponding end, as is denoted with a dashed line 72 in the figure.

In a still further embodiment of the invention, the relative weight of the frequencies around the pulse rate of the subject may be enhanced, as is shown in FIG. 8. As in the case of the notch, the form, height, and width of the positive offset 80 locating around the pulse rate may vary. Similarly as the respiration rate, the pulse rate may be determined for the weight function by any appropriate method and various methods may be used depending on the equipment available.

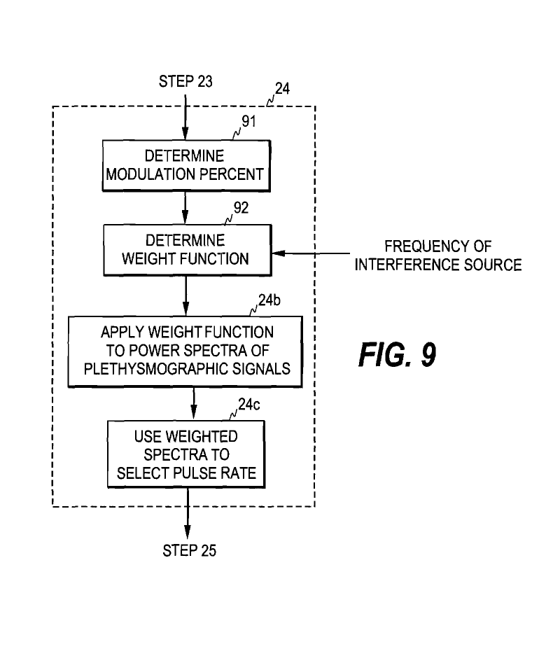

In further embodiments of the invention, the weighting of the power spectrum values locating between the lower limit and the upper limit, i.e. within the passband, is carried out conditionally or adaptively depending on the amplitude of the arterial pulsation. In one embodiment of conditional weighting, no weighting of the respiration frequency or its harmonic frequencies is employed if the amplitude of the arterial pulsation exceeds a predetermined threshold, i.e. if the modulation percent (percentual ratio of the AC and DC components) is above a predetermined threshold, such as 0.5. This embodiment is illustrated in FIG. 9. As compared to the embodiment of FIG. 6, this embodiment now comprises an additional step 91, in which the modulation percentage is determined. Furthermore, the determination of the weight function is denoted with reference numeral 92 in the embodiment of FIG. 9, since the content of the step dffers from the corresponding step 24a in the embodiment of FIG. 6. In step 92, the modulation percentage obtained is compared with a predetermined threshold. If the modulation percentage is below the threshold, the weighting of the power spectrum values is carried out as discussed in connection with FIGS. 6 to 8. However, if the modulation percentage is greater than or equal to the threshold, the weighting of the respiration frequency and/or its harmonic frequencies is skipped, if the said frequencies locate within the passband. In this case, the pulse rate is selected based on power spectrum values weighted according to a weight function in which the relative weight is constant, preferably one, in the range between the lower limit and the upper limit, and zero outside this range. In other words, if the modulation percentage is greater than or equal to the threshold, the weight function is similar to the one which is normally used in the embodiments of FIGS. 6 to 8 when the respiration frequency and/or its harmonic frequencies are outside the passband.

In adaptive weighing, the characteristics of the notch depend on the modulation percent. For example, the depth of the notch 71 may be inversely proportional to the modulation percent.

Conditional and adaptive weighting may also be combined. For example, if the modulation percent is below 0.5, the amplitudes the respiration frequency and its harmonic frequencies which locate within the passband could be lowered by P percent, where

P=100−200*Mod %, where Mod % is the modulation percent.

In all embodiments of the invention, the determination of the ratio R may be performed based on the weighted or unweighted spectral values, since the ratio of the spectral values in equation (2) remains the same if the weighting is similar for all wavelengths.

A conventional monitoring device, such as pulse oximeter, may also be upgraded by providing it with a new software module or a plug-in software module that enables the device to weigh the PSD values according to one of the above embodiments. The software module may be delivered, for example, on a data carrier, such as a CD or a memory card, or via a telecommunications network. The software processing the plethysmographic signal data may be divided into four logical portions according its operation: a first program code portion is configured to derive power spectnjms for the plethysmographic signals obtained from the subject, a second program code portion is configured to determine a weight function whose characteristics depend on the frequency of an interference source, a third program code portion is configured to apply the weight function to the power spectrums, and a fourth program code portion is configured to define a blood related parameter based on the weighted power spectrums. A plug-in software module may comprise the above second and third program code portions, since a conventional SpO₂ algorithm is already configured to determine the power spectrums of the plethysmographic signals obtained from the subject and to define the blood related parameter based on the power spectrums.

Although the invention was described above with reference to the examples shown in the appended drawings, it is obvious that the invention is not limited to these, but may be modified by those skilled in the art without departing from the scope and spirit of the invention. As discussed above, the number of blood related signals may vary depending on the application. Furthermore, all blood related signals obtained from the subject are not necessarily employed for the determination of the blood related parameter. A plurality of interference frequencies may also be measured from one or more interference sources.

1. A method for assessing a blood related parameter of a subject, the method comprising:
obtaining at least one blood related signal from a subject;
deriving a power spectrum for at least one of the at least one blood related signal, thereby to obtain at least one power spectrum;
identifying at least one interference frequency of at least one interference source causing unwanted modulation in the at least one blood related signal;
determining a weight function in which the relative weight of at least one frequency is dampened, the at least one frequency depending on the at least one interference frequency identified;
applying the weight function to at least one of the at least one power spectrum, thereby to obtain at least one weighted power spectrum; and
defining the blood related parameter based on the at least one weighted power spectrum.

2. A method according to claim 1, wherein
the obtaining includes obtaining at least two plethysmographic signals from the subject;
the applying includes applying the weight function to at least two power spectrums, thereby to obtain at least two weighted power spectrums; and
the defining includes defining the blood related parameter based on the at least two weighted power spectrums, in which the blood related parameter represents arterial oxygen saturation.

3. A method according to claim 1, wherein the identifying includes identifying the at least one interference frequency of the at least one interference source, in which the at least one interference frequency represents the respiration rate of the subject.

4. A method according to claim 3, wherein the determining includes determining the weight function, in which the weight function comprises a notch having a minimum substantially at the respiration rate.

5. A method according to claim 1, wherein the determining includes determining the weight function, and wherein the relative weights of frequencies around the pulse rate of the subject are enhanced in the weight function.

6. A method according to claim 3, further comprising determining modulation percent for at least one of at least one blood related signal.

7. A method according to claim 6, wherein the determining includes determining the weight function, and wherein
the relative weights of frequencies below a lower limit, beyond an upper limit, and around the respiration rate are dampened in the weight function, if the modulation percent fulfills a predetermined criterion and
the relative weights of frequencies below the lower limit and beyond an upper limit are dampened in the weight function, if the modulation percent fails to fulfill the predetermined criterion.

8. A method according to claim 6, wherein the determining includes determining the weight function, in which the weight function comprises a notch having a minimum at the respiration rate, and wherein the depth of the notch is inversely proportional to the modulation percent.

9. A method according to claim 7, wherein the determining includes determining the weight function, in which the weight function comprises a notch having a minimum at the respiration rate, and wherein the depth of the notch is inversely proportional to the modulation percent.

10. A method according to claim 7, wherein the identifying includes deriving the respiration rate from at least one of the at least one blood related signal.

11. A method according to claim 7, wherein the identifying includes deriving the respiration rate from a further physiological signal.

12. A method according to claim 1, wherein the determining includes determining the weight function, and wherein the at least one frequency comprises a harmonic frequency of the at least one interference frequency.

13. A method according to claim 1, wherein the determining includes determining the weight function, and wherein
the relative weights of frequencies below a predetermined lower frequency limit and beyond a predetermined upper frequency limit are dampened in the weight function, if the at least one interference frequency is below the lower limit;
the relative weights of frequencies below the predetermined lower frequency limit, beyond the predetermined upper frequency limit and around at least one of the at least one interference frequency are dampened in the weight function, if any of the at least one interference frequency is between the predetermined lower frequency limit and the predetermined upper frequency limit.

14. A method according to claim 1, wherein the defining includes defining the blood related parameter based on the at least one weighted power spectrum, in which the blood related parameter represents the pulse rate of the subject.

15. An apparatus for assessing a blood related parameter of a subject, the apparatus comprising:
a measurement unit configured to measure at least one blood related signal from a subject;
a transform unit configured to derive a power spectrum for at least one of the at least one blood related signal, thereby to obtain at least one power spectrum;
a frequency identification unit configured to identify at least one interference frequency of at least one interference source causing unwanted modulation in the at least one blood related signal;
a first calculation unit configured to determine a weight function in which the relative weight of at least one frequency is dampened, the at least one frequency depending on the at least one interference frequency;
a weighting unit configured to apply the weight function to at least one of the at least one power spectnjm, thereby to obtain at least one weighted power spectrum; and
a second calculation unit configured to define the blood related parameter based on the at least one weighted power spectrum.

16. An apparatus according to claim 15, wherein
the weighting unit is configured to apply the weight function to at least two power spectrums, thereby to obtain at least two weighted power spectrums; and
the second calculation unit is configured to define arterial oxygen saturation of the subject based on the at least two weighted power spectrums.

17. An apparatus according to claim 15, wherein the frequency identification unit is configured to determine the respiration rate of the subject.

18. An apparatus according to claim 15, wherein the weight function comprises a notch substantially at a frequency selected from a group including the at least one interference frequency of the at least one interference source and harmonic frequencies thereof.

19. An apparatus according to claim 15, wherein the relative weights of frequencies around the pulse rate of the subject are enhanced in the weight function.

20. An apparatus according to claim 17, further comprising a third calculation unit configured to determine modulation percent for at least one of at least one blood related signal.

21. An apparatus according to claim 20, wherein the relative weights of frequencies below a predetermined lower frequency limit, beyond a predetermined upper frequency limit, and around the at least one interference frequency of the at least one interference source are dampened in the weight function, if the modulation percent fulfills a predetermined criterion and
the relative weights of frequencies below the predetermined lower frequency limit and beyond the predetermined upper frequency limit are dampened in the weight function, if the modulation percent fails to fulfill the predetermined criterion.

22. An apparatus according to claim 20, wherein the weight function comprises a notch having a minimum substantially at the respiration rate, and wherein the depth of the notch is inversely proportional to the modulation percent.

23. An apparatus according to claim 21, wherein the weight function comprises a notch having a minimum substantially at the respiration rate, and wherein the depth of the notch is inversely proportional to the modulation percent.

24. An apparatus to claim 17, wherein the frequency identification unit is configured to derive the respiration rate from at least one of the at least one blood related signal.

25. An apparatus according to claim 17, wherein the frequency identification unit is configured to identify the respiration rate from a further physiological signal.

26. An apparatus according to claim 15, wherein the second calculation unit is configured to define the pulse rate of the subject based on the at least one weighted power spectrum.

27. An apparatus for assessing a blood related parameter of a subject, the apparatus comprising:
measurement means for measuring at least one blood related signal from a subject;
transform means for deriving a power spectrum for at least one of the at least one blood related signal, thereby to obtain at least one power spectrum;
frequency identification means for identifying at least one interference frequency of at least one interference source causing unwanted modulation in the at least one blood related signal;
first calculation means for determining a weight function in which the relative weight of at least one frequency is dampened, the at least one frequency depending on the at least one interference frequency;
weighting means for applying the weight function to at least one of the at least one power spectrum, thereby to obtain at least one weighted power spectrum; and
second calculation means for defining the blood related parameter based on the at least one weighted power spectrum.

28. A computer program product for an apparatus configured to assess a blood related parameter of a subject, the computer program product comprising:
a first program code portion configured to determine a weight function in which the relative weight of at least one frequency is dampened, the at least one frequency depending on at least one frequency of an interference source causing unwanted modulation in at least one blood related signal; and
a second program code portion configured to apply the weight function to at least one power spectrum of the at least one blood related signal, thereby to obtain at least one weighted power spectrum.

29. A computer program product according to claim 28, further comprising:
a third program code portion configured to derive the at least one power spectrum;
a fourth program code portion configured to define the blood related parameter based on the at least one weighted power spectrum.
